(19) United States
(12) Patent Application Publication
(10) Pub. No.: US 2012/0211662 A1
(43) Pub. Date: Aug. 23, 2012

(54) RADIOGRAPHIC IMAGE CAPTURING APPARATUS

(75) Inventors: Shou SHIMIZUKAWA, Kanagawa-ken (JP); Kiyoshi KONDOU, Kanagawa-ken (JP)

(73) Assignee: FUJIFILM CORPORATION, Tokyo (JP)

(21) Appl. No.: 13/358,333

(22) Filed: Jan. 25, 2012

(30) Foreign Application Priority Data
Feb. 21, 2011 (JP) 2011-035218

(51) Int. Cl. G01T 1/20; G01T 1/00

(52) U.S. Cl. 250/369; 250/336.1

(57) ABSTRACT

A radiographic image capturing apparatus includes a radiation conversion panel for converting radiation into radiographic images, an A/D converter for performing an A/D conversion process on image signals depending on the radiographic image output from the radiation conversion panel, and a number-of-sampling determiner for determining a number of times of sampling for the A/D conversion process performed by the A/D converter, based on the dose of radiation, which is applied to the radiation conversion panel.

### CROSS-REFERENCE TO RELATED APPLICATION

This application is based upon and claims the benefit of priority from Japanese Patent Application No. 2011-035218 filed on Feb. 21, 2011, of which the contents are incorporated herein by reference.

### BACKGROUND OF THE INVENTION

1. Field of the Invention

The present invention relates to a radiographic image capturing apparatus, which incorporates therein a radiation conversion panel for converting radiation into a radiographic image and an A/D converter for converting image signals representing the radiographic image from an analog format into a digital format.

2. Description of the Related Art

In the medical field, it has been customary to use a radiographic image capturing apparatus (hereinafter referred to as an “electronic cassette”) for capturing radiographic images of a subject by detecting radiation that has passed through the subject. To make the electronic cassette portable, the electronic cassette incorporates therein a power supply including an electric energy storage unit such as a battery or the like. The electronic cassette converts an image signal representing the radiographic image from an analog format into a digital format.

The portable electronic cassette may be designed according to the technology disclosed in Japanese Laid-Open Patent Publication No. 07-275235, for acquiring a radiographic image of digital data, which is of high quality with reduced random noise by converting a radiographic image in the form of analog data into a radiographic image in the form of digital data, according to an oversampling process based on image capturing conditions including a tube voltage and a tube current of a radiation source that emits radiation, the thickness of the subject, an image capturing area, etc.

### SUMMARY OF THE INVENTION

Upon conversion of the analog radiographic image into a digital radiographic image according to the oversampling process, since the oversampling process takes a long total period of time, the oversampling process causes quick power consumption, and the portable electronic cassette suffers from a tradeoff between efforts to increase quality of radiographic images and power consumption. Furthermore, in actual image capturing processes, since a dose of radiation depending on an image capturing menu is applied through the subject to a radiation conversion panel, it is necessary to perform a sampling process (A/D conversion process) depending on the dose of radiation applied to the radiation conversion panel.

It is an object of the present invention to provide a radiographic image capturing apparatus, which is capable of capturing radiographic images of high quality while minimizing power consumption, and which also is capable of performing an A/D conversion process depending on the dose of radiation applied to a radiation conversion panel.

To achieve the above object, there is provided in accordance with the present invention a radiographic image capturing apparatus comprising a radiation conversion panel for converting radiation into a radiographic image, an A/D converter for performing an A/D conversion process on an image signal depending on the radiographic image output from the radiation conversion panel, and a number-of-sampling determiner for determining a number of times of sampling for the A/D conversion process performed by the A/D converter, based on the dose of radiation that is applied to the radiation conversion panel.

According to the present invention, since a number of times of sampling for the A/D conversion process performed by the A/D converter is determined based on the dose of radiation applied to the radiation conversion panel, in the case that the A/D converter performs an A/D conversion process on the image signal at a determined number of times of sampling, the A/D conversion process is performed depending on the dose of radiation, such that high-quality radiographic images can be acquired while minimizing power consumption.

The radiographic image capturing apparatus further includes a signal reader for reading out the image signal from the radiation conversion panel, and a leak signal detector for detecting leak signals, which leak from the radiation conversion panel from a time at which radiation starts to be applied to the radiation conversion panel until a time at which the image signal is read out from the radiation conversion panel by the signal reader. The number-of-sampling determiner estimates the dose of radiation based on the signal levels of the leak signals, which are detected by the leak signal detector, and determines the number of times of sampling based on the estimated dose.

The leak signals are signals that leak from the radiation conversion panel depending on the signal level of the image signal before the signal reader reads out the image signal. Since the signal level of the image signal depending on the dose of radiation is greater, the signal levels of the leak signals are also higher. Therefore, the dose of radiation can be estimated from the signal levels of the leak signals, and an A/D conversion process suitable for the dose of radiation can be performed, assuming that the number of times of sampling is determined based on the estimated dose of radiation.

If the signal levels of the leak signals are relatively low, the number-of-sampling determiner may control the A/D converter to perform an oversampling A/D conversion process, and if the signal levels of the leak signals are relatively high, the number-of-sampling determiner may control the A/D converter to cancel the oversampling A/D conversion process.

In other words, if the signal levels of the leak signals are low, then the signal level of the image signal also is low, and the dose of radiation is small. In this case, random noise can be reduced by performing the oversampling A/D conversion process on the image signal.

If the signal levels of the leak signals are high, then the signal level of the image signal also is high, and the dose of radiation is large. In this case, inasmuch as a radiographic image of a low noise level can be generated without requiring the oversampling A/D conversion process, the oversampling A/D conversion process can be canceled. As a result, undue power consumption can be avoided.

The radiation conversion panel comprises a scintillator for converting the radiation into visible light, and a photoelectric transducer layer for converting the visible light into the image signal. The photoelectric transducer layer includes a matrix of photoelectric transducer elements for converting the visible light into the image signal, and switching elements for outputting the image signal from the photoelectric transducer elements to the signal reader, a plurality of scanning lines that are supplied with control signals for turning on the switching elements, and a plurality of signal lines extending across the scanning lines, which are supplied with the image signal output from the photoelectric transducer elements. The switching elements are connected to the scanning lines and to the signal lines.

Specific arrangements (1) through (4) for detecting the leak signals will be described below.

(1) If the switching elements are in an off state, the leak signals are signals that leak from the photoelectric transducer elements via the switching elements and the signal lines into the signal reader. The leak signal detector may detect the leak signals, which leak from the signal lines into the signal reader, and generate a profile representative of changes in the signal levels of the leak signals along the array of signal lines. The number-of-sampling determiner may determine the number of times of sampling based on the generated profile.

Since the image signal is read out via the signal lines into the signal reader, random noise can reliably be reduced by determining the number of times of sampling according to the profile, and by performing the A/D conversion process on the read image signal at the determined number of times of sampling. As a result, high-quality radiographic images can reliably be acquired.

The number-of-sampling determiner may calculate a level of difference between maximum and minimum values of the signal levels in the profile, and determine the number of times of sampling based on the calculated level difference. Since the signal level of the image signal differs between the signal lines, it is possible to acquire high-quality radiographic images accurately by determining the number of times of sampling based on the level difference, and by performing the A/D conversion process on the read image signal at the determined number of times of sampling.

The signal reader may include amplifiers connected between the signal lines and the A/D converter. If the leak signals leak from the signal lines into the signal reader, the amplifiers should preferably comprise variable-gain amplifiers for amplifying the leak signals with a gain that is higher than a gain for amplifying the image signal.

Since the leak signals are lower in signal level than the image signal, upon acquiring the leak signals, the gain of the variable-gain amplifiers may be increased, and the leak signals may be amplified by the variable-gain amplifiers at such an increased gain. In this manner, the number of times of sampling can be determined accurately.

(2) The radiation conversion panel may include certain switching elements that are introduced at the time the radiation conversion panel is manufactured, the switching functions of which do not operate normally, i.e., which are in an on state at all times. In this case, the photoelectric transducer elements and the signal lines that are connected to such switching elements make up components (defects), which do not contribute to detecting and reading out image signals from the photoelectric transducer elements.

According to the present invention, if such defects occur in the radiation conversion panel, the leak signal detector may detect only leak signals, which leak from the signal lines connected to the switching elements into the signal reader.

Since the above defects are used as components dedicated to the detection of leak signals, all of the components of the radiation conversion panel can be used effectively, and other components, which are dedicated only to the detection of leak signals, do not need to be added.

(3) The photoelectric transducer layer may include other photoelectric transducer elements for converting the visible light into electric signals, and a leak signal output line for outputting the electric signals as the leak signals from the other photoelectric transducer elements to the signal reader. The leak signal detector may detect only the leak signals that are output from the leak signal output line to the signal reader.

According to arrangement (3), unlike arrangement (2), the photoelectric transducer element and the leak signal output line (dedicated line), which detect and read out the leak signals, are positively included in the photoelectric transducer layer. Although such components serve as components dedicated to the detection of leak signals, the components are capable of detecting leak signals regardless of whether the image signal is being read out.

(4) The radiographic image capturing apparatus may further comprise bias lines for supplying a bias voltage to the photoelectric transducer elements, a bias power supply for applying the bias voltage via the bias lines to the photoelectric transducer elements, and a current detector for detecting bias currents flowing through the bias lines. The leak signal detector detects a change in the bias currents, which occurs upon application of radiation to the radiation conversion panel, as a change in the signal levels of the leak signals.

Attention is paid to the fact that the bias currents are changed by application of radiation to the radiation conversion panel. A change in the bias currents is regarded as a change in the signal levels of the leak signals and is detected, for thereby efficiently detecting the leak signals.

The photoelectric transducer layer may include a plurality of bias lines extending across the scanning lines, and the current detector may comprise a plurality of current detectors connected respectively to the bias lines. The leak signal detector may detect a change in the bias currents, which flow through the bias lines, and which are detected by the current detectors, respectively, as a change in the signal levels of the leak signals.

Since the current detectors detect leak signals on the respective bias lines, it is possible to accurately reduce random noise in the image signal from the photoelectric transducer elements.

The above and other objects, features, and advantages of the present invention will become more apparent from the following description when taken in conjunction with the accompanying drawings in which preferred embodiments of the present invention are shown by way of illustrative example.

### BRIEF DESCRIPTION OF THE DRAWINGS

FIG. 1 is a schematic view, partially in block form, of a radiographic image capturing system incorporating therein an electronic cassette according to a first embodiment of the present invention;

FIG. 2 is a perspective view of the electronic cassette shown in FIG. 1;

FIG. 3 is a block diagram of the electronic cassette shown in FIG. 1;

FIG. 4 is a flowchart of an operation sequence of the electronic cassette shown in FIG. 1 for capturing a radiographic image of a subject;

FIG. 5 is a timing chart showing a time sequence from application of radiation until reading of a radiographic image;

FIG. 6 is a diagram illustrative of a profile of a leak signal along an array of signal lines;

FIG. 7 is a block diagram of an electronic cassette according to a second embodiment of the present invention;

FIG. 8 is a block diagram of an electronic cassette according to a third embodiment of the present invention;

FIG. 9 is a block diagram of an electronic cassette according to a fourth embodiment of the present invention;

FIG. 10 is a timing chart showing the manner in which a voltage value changes with time depending on a bias current detected by a current detector shown in FIG. 9; and

FIG. 11 is a block diagram of a portion of an electronic cassette according to a fifth embodiment of the present invention.

### DESCRIPTION OF THE PREFERRED EMBODIMENTS

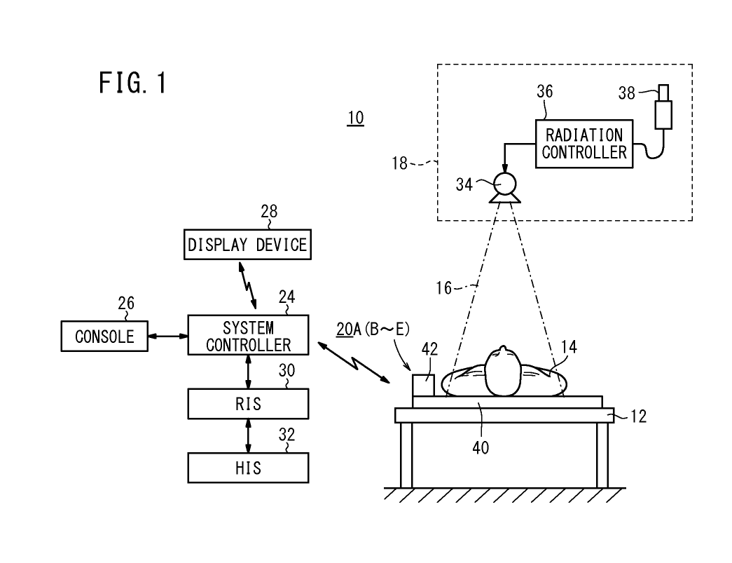

Radiographic image capturing apparatus according to preferred embodiments of the present invention will be described in detail below with reference to FIGS. 1 through 11.

### Configuration of the First Embodiment

FIG. 1 is a schematic view, partially in block form, of a radiographic image capturing system 10 incorporating therein an electronic cassette 20A as a radiographic image capturing apparatus according to a first embodiment of the present invention.

As shown in FIG. 1, the radiographic image capturing system 10 includes a radiation device 18 for applying radiation 16 to a subject 14, i.e., a patient lying on an image capturing base 12 such as a bed or the like, an electronic cassette (radiographic image capturing apparatus) 20A for detecting radiation 16 that has passed through the subject 14 and converting the detected radiation into a radiographic image, a system controller 24 functioning as an image processor, which controls the radiographic image capturing system 10 in its entirety, a console 26 for receiving input actions from a doctor or a radiological technician as the user, and a display device 28 for displaying captured radiographic images.

The system controller 24, the electronic cassette 20A, and the display device 28 send signals to and receive signals from each other by way of a wireless LAN (Local Area Network) according to standards such as UWB (Ultra-Wide Band), IEEE802.11a/b/g/n, or the like, or wireless communications using milliwaves. The system controller 24, the electronic cassette 20A, and the display device 28 may also send signals to and receive signals from each other by way of wired communications using cables.

The system controller 24 is connected to a radiology information system (RIS) 30, which generally manages radiographic image information handled by the radiological department of a hospital together with other information. The RIS 30 is connected to a hospital information system (HIS) 32, which generally manages medical information in the hospital.

The radiation device 18 has a radiation source 34 for emitting radiation 16, a radiation controller 36 for controlling the radiation source 34, and a radiation switch 38. The radiation source 34 applies radiation 16 through the subject 14 toward the electronic cassette 20A. Radiation 16 that is emitted from the radiation source 34 may be X-rays, α-rays, β-rays, γ-rays, an electron beam, or the like. The radiation switch 38 can be pushed in two strokes, i.e., in a half stroke and a full stroke. If the radiation switch 38 is pushed in a half stroke by the user, the radiation switch 38 sends a signal to the radiation controller 36 to prepare the radiation source 34 to emit radiation 16. If the radiation switch 38 is subsequently pushed in a full stroke, the radiation switch 38 sends a signal to the radiation controller 36 to enable the radiation source 34 to start emitting radiation 16. The radiation controller 36 has an input device, not shown, which is operated by the user to set values for the irradiation time of the radiation 16, a tube voltage, and a tube current of the radiation source 34, etc. The radiation controller 36 controls the radiation source 34 to emit radiation 16 based on the irradiation time, the tube voltage, the tube current, etc., which have been set using the input device.

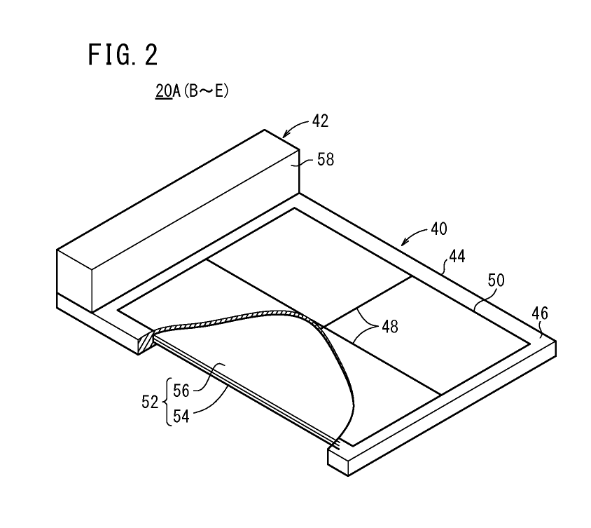

FIG. 2 is a perspective view of the electronic cassette 20A shown in FIG. 1.

As shown in FIG. 2, the electronic cassette 20A is a portable electronic cassette including a panel 40 disposed between the image capturing base 12 (see FIG. 1) and the subject 14, and a controller 42 disposed on the panel 40. The panel 40 is thinner than the controller 42.

The panel 40 includes a substantially rectangular housing 44 made of a material permeable to radiation 16. The housing 44 has an upper surface serving as an irradiated surface 46, which is irradiated with radiation 16. The irradiated surface 46 has guide lines 48 indicative of an image capturing area and an image capturing position for the subject 14. The guide lines 48 include an outer frame representing an image capturing area 50, which indicates a maximum irradiation range (irradiation field) to be irradiated with radiation 16 on the irradiated surface 46. The guide lines 48 have a central position where the guide lines 48 cross each other in a crisscross pattern, which is indicative of the central position of the image capturing area 50.

The housing 44 houses therein a radiation conversion panel 52 for converting radiation 16 that has passed through the subject 14, and which has entered the housing 44, into a radiographic image. The radiation conversion panel 52 is an indirect conversion radiation detector, which includes a scintillator 54 for converting radiation 16 into electromagnetic waves of another wavelength, such as visible light, and a photoelectric transducer layer 56 for converting the electromagnetic waves produced by the scintillator 54 into electric signals.

In FIG. 2, the radiation conversion panel 52 is illustrated as an ISS (Irradiation Side Sampling) radiation detector of a face-side reading type, wherein the photoelectric transducer layer 56 and the scintillator 54 are successively arranged in this order along the direction in which radiation 16 is applied. However, the radiation conversion panel 52 may be an RSS (Penetration Side Sampling) radiation detector of a reverse-side reading type, wherein the scintillator 54 and the photoelectric transducer layer 56 are successively arranged in this order along the direction in which radiation 16 is applied. The scintillator 54 may be made of cesium iodide (CsI) or gadolinium oxide sulfur (GOS). Alternatively, the radiation conversion panel 52 may be a direct conversion radiation detector, which is free of a scintillator, for directly converting radiation 16 into electric signals. In the present embodiment, it is assumed that the radiation conversion panel 52 is an ISS radiation detector.

The controller 42 includes a substantially rectangular housing 58 made of a material impermeable to radiation 16. The controller 42 is disposed outside of the image capturing area 50 of the housing 44. Since the housing 58 may be disposed in a location that is not irradiated with radiation 16, the controller 42 may be positioned within the image capturing area 50, provided that the location is not irradiated with radiation 16 and does not interfere with the subject 14. Instead of providing the controller 42 at a separate location on the housing 44, the housing 44 may have a protrusive region, which incorporates the controller 42 therein.

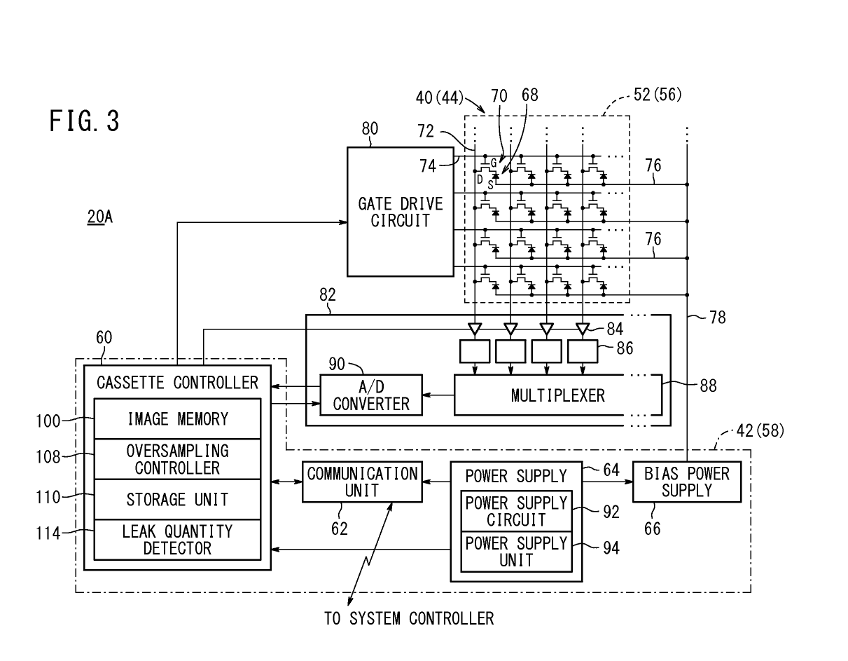

FIG. 3 is a block diagram of the electronic cassette 20A.

As shown in FIG. 3, the controller 42 has a cassette controller 60 for controlling the electronic cassette 20A in its entirety, a communication unit 62 for sending signals to and receiving signals from the system controller 24 and the radiation device 18 via a wireless communication link, a power supply 64 for supplying electric energy to the electronic cassette 20A, and a bias power supply 66 for energizing the photoelectric transducer layer 56.

The photoelectric transducer layer 56 comprises a plurality of photoelectric transducer elements 68, such as pin-type photodiodes or phototransistors for converting radiation 16 into electric charges and storing the electric charges, and a plurality of thin film transistors (TFTs) 70, which are connected as switching elements to the respective photoelectric transducer elements 68. In FIG. 3, the photoelectric transducer elements 68 are illustrated as pin-type photodiodes.

The photoelectric transducer layer 56 also includes a plurality of signal lines 72 and a plurality of gate lines (scanning lines) 74, which are disposed on one surface of a substrate made up of glass or a synthetic resin. The signal lines 72 and the gate lines 74 extend across each other, defining small areas in which respective pairs of photoelectric transducer elements 68 and TFTs 70 are disposed. Therefore, the photoelectric transducer elements 68 and the TFTs 70 are arranged on the substrate in a two-dimensional matrix. Photoelectric transducer elements 68 in each row are connected to a single bias line 76, and the bias lines 76 are connected to a single common line 78.

The photoelectric transducer elements 68 have respective anode electrodes connected to the bias lines 76, and respective cathode electrodes connected to respective source electrodes S of the TFTs 70. The TFTs 70 have respective gate electrodes G connected by gate lines 74 to a gate drive circuit 80, and respective drain electrodes D connected by signal lines 72 to a signal readout circuit (signal reader) 82.

The bias power supply 66 applies a reverse bias voltage to the photoelectric transducer elements 68 via the common line 78 and the bias lines 76. In FIG. 3, the bias lines 76 are connected through the anode electrodes to p-layers of the pin-type photoelectric transducer elements 68. Therefore, the bias power supply 66 applies a negative voltage, via the common line 78 and the bias lines 76, as a reverse bias voltage to the anode electrodes of the photoelectric transducer elements 68. The negative voltage may be a voltage, which is lower by a prescribed voltage than the voltage at the cathode electrodes of the photoelectric transducer elements 68. If the pin layers of the photoelectric transducer elements 68 are reversed, i.e., if the photoelectric transducer elements 68 are opposite in polarity, and the bias lines 76 are connected to the cathode electrodes of the photoelectric transducer elements 68, then the bias power supply 66 applies a positive voltage as a reverse bias voltage to the cathode electrodes of the photoelectric transducer elements 68. The positive voltage may be a voltage, which is lower by a prescribed voltage than the voltage at the anode electrodes of the photoelectric transducer elements 68. In this case, the photoelectric transducer elements 68 are connected in a reverse manner to the bias power supply 66 shown in FIG. 3.

In the case that the gate drive circuit 80 applies a signal readout voltage (control signal) to the gate electrodes G of the TFTs 70 via the gate lines 74, the gates of the TFTs 70 are opened, thereby reading out the electric charges stored in the photoelectric transducer elements 68, i.e., electric signals (an image signal), via the source electrodes S and the drain electrodes D of the TFTs 70 into the signal lines 72.

The signal readout circuit 82 includes a plurality of amplifiers 84 connected respectively to the signal lines 72, a plurality of sample and hold circuits 86 connected respectively to the amplifiers 84, a multiplexer 88 connected to the sample and hold circuits 86, and an A/D converter 90 connected to the multiplexer 88. Electric signals, which are read out from the columns of photoelectric transducer elements 68 via the signal lines 72, are amplified by the amplifiers 84, each of which are of a variable gain type in the form of a charge amplifier, and then the electric signals are sampled and held by the sample and hold circuits 86. The sample and hold circuits 86 supply sampled and held signals to the multiplexer 88, which supplies multiplexed signals to the A/D converter 90. The A/D converter 90 converts the multiplexed signals into digital signals (digital values). The A/D converter 90 outputs the digital signals, which represent electric signals from the photoelectric transducer elements 68, to the cassette controller 60.

The power supply 64 includes a power supply circuit 92 and a power supply unit (electric energy storage unit) 94. The power supply unit 94 is an electric energy storage means such as a battery, a capacitor, or the like. The power supply circuit 92 is a power converter circuit such as a DC/DC converter or the like for converting a voltage from the power supply unit 94 into desired voltages, and applying the desired voltages to various components of the electronic cassette 20A.

The cassette controller 60 is a computing system made up of a microcomputer including a CPU, not shown, which performs various functions by reading and executing programs stored in a ROM.

More specifically, the cassette controller 60 has an image memory 100, an oversampling controller (number-of-sampling determiner) 108, a storage unit 110, and a leak quantity detector (leak signal detector) 114.

The image memory 100 stores radiographic images acquired from the radiation conversion panel 52. The storage unit 110 stores cassette ID information for identifying the electronic cassette 20A.

The leak quantity detector 114 detects leak currents (hereinafter referred to as “leak signals”), which leak from the radiation conversion panel 52 during a time interval from the start of application of radiation 16 to the subject 14 until a radiographic image of the subject 14 is captured, i.e., from the start of application of radiation 16 to the radiation conversion panel 52 until electric signals from the radiation conversion panel 52 start to be read out by the signal readout circuit 82.

Leak currents (leak signals) refer to currents (signals) generated upon leakage of a portion of the electric charges stored in the photoelectric transducer elements 68 into the signal readout circuit 82 via the TFTs 70 and the signal lines 72, in a case where radiation 16 is applied to the radiation conversion panel 52 while all of the TFTs 70 are in an off state.

If the dose of radiation 16 applied to the radiation conversion panel 52 is greater, then the amount of electric charge stored in the photoelectric transducer elements 68 becomes greater. Conversely, if the dose of radiation 16 applied to the radiation conversion panel 52 is smaller, then the amount of electric charge stored in the photoelectric transducer elements 68 becomes smaller. The TFTs 70 are turned off if the switching resistance, i.e., the electric resistance between the source electrodes S and the drain electrodes D thereof, is high, and the TFTs are turned on if the switching resistance is low.

Since the TFTs 70 are turned on or off depending on the value of the switching resistance, if the amount of electric charge stored in the photoelectric transducer elements 68 is large, even though the value of the switching resistance is high enough to turn off the TFTs 70, then the signal levels of leak signals, i.e., the amount of leak current leaking from the photoelectric transducer elements 68 into the signal readout circuit 82, are large. If the amount of electric charge stored in the photoelectric transducer elements 68 is small, then the signal levels of the leak signals leaking from the photoelectric transducer elements 68 into the signal readout circuit 82 are small.

Inasmuch as the amount of electric charge stored in the photoelectric transducer elements 68 depends on the dose of radiation 16 applied to the radiation conversion panel 52, the signal level of the leak signals also depends on the dose of radiation 16 applied to the radiation conversion panel 52. Since the leak signals are signals that leak into the signal readout circuit 82 while the TFTs 70 are in an off state, the signal levels thereof are much lower than the signal levels of electric signals that are read into the signal readout circuit 82 while the TFTs 70 are in an on state.

The leak quantity detector 114 sets the gain of the amplifiers 84 to a level higher than the gain at the time that the electric signals are read out, during the time interval at which radiation 16 starts to be applied to the radiation conversion panel 52 until the time at which electric signals from the radiation conversion panel 52 start to be read out by the signal readout circuit 82. Therefore, the leak currents, which have been amplified to a high level by the amplifiers 84, are sampled and held by the sample and hold circuits 86, and then the leak currents are converted into digital signals by the A/D converter 90. As a result, the leak quantity detector 114 can easily detect (digital data of) the leak signals, the signal level of which has been amplified significantly.

Since plural photoelectric transducer elements 68 are connected to each signal line 72 through respective TFTs 70, each signal line 72 transmits the sum of the leak signals from the plural photoelectric transducer elements 68 to the signal readout circuit 82. Therefore, the leak quantity detector 114 detects the signal level of the sum of the leak signals from each signal line 72. After having detected the signal level of the sum of the leak signals from each signal line 72, the leak quantity detector 114 changes the gain of the amplifiers 84 to the original gain thereof, i.e., a gain for reading out electric signals, in preparation for reading out electric signals from the photoelectric transducer elements 68.

The oversampling controller 108 estimates the dose of radiation 16 from the signal levels of the leak signals, which are detected by the leak quantity detector 114, determines a number of times of sampling (also simply referred to as “number of sampling”) for the A/D conversion process of the A/D converter 90 based on the estimated dose, and controls the A/D converter 90 in order to convert electric signals into digital signals at a predetermined number of sampling. More specifically, the oversampling controller 108 decides whether an oversampling A/D conversion process should be performed or canceled based on the estimated dose, and determines the number of sampling depending on such a decision.

### Operations of the First Embodiment

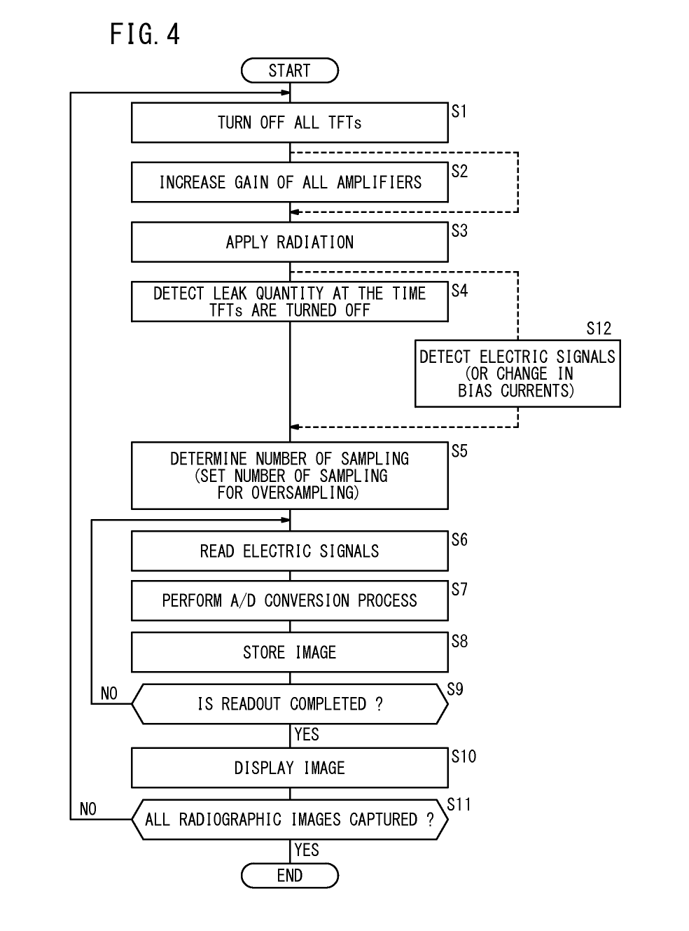

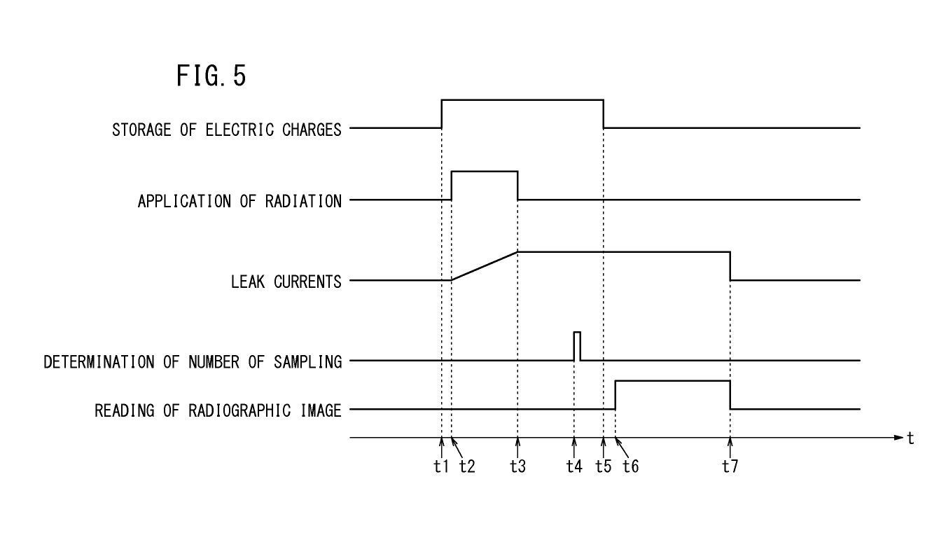

Operations of the radiographic image capturing system 10 incorporating therein the electronic cassette 20A according to the first embodiment will be described below with reference to the flowchart shown in FIG. 4 and the timing chart shown in FIG. 5, as well as with reference to FIGS. 1 through 3.

First, the user adjusts the distance between the radiation source 34 and the radiation conversion panel 52 to a certain SID (subject-to-image distance), and positions the subject 14 such that the subject 14 is disposed over the irradiated surface 46 with a region to be imaged of the subject 14 being located within the image capturing area 50, and such that the center of the subject 14 is aligned substantially with the central position of the image capturing area 50.

Then, the user operates the console 26 to select the region to be imaged of the subject 14. The system controller 24 sets image capturing conditions depending on the selected region to be imaged of the subject 14. At this time, the system controller 24 may display an image of the region to be imaged of the subject 14 on the display device 28, so that the user can select the region to be imaged of the subject 14 while observing the displayed image. Upon the user selecting a region to be imaged of the subject 14, the system controller 24 may display an image of the selected region together with the image capturing conditions, thereby allowing the user to confirm the image capturing condition details.

Then, the system controller 24 sends a startup signal to the communication unit 62 of the electronic cassette 20A via a communication unit. The power supply 64 of the electronic cassette 20A continuously supplies electric power to the cassette controller 60 and the communication unit 62. Upon reception of the startup signal by the communication unit 62, the cassette controller 60 controls the power supply 64 to supply electric power to the bias power supply 66. The bias power supply 66 applies a reverse bias voltage to the photoelectric transducer elements 68, so as to make the photoelectric transducer elements 68 capable of storing electric charges. If a plurality of radiographic images of the subject 14 are to be captured, then along with the startup signal, the system controller 24 may send an instruction signal to the communication unit 62 in order to instruct capturing of such radiographic images.

In order for the radiation source 34 to apply radiation 16 under the set image capturing conditions, the user operates an input device (not shown) of the radiation controller 36, so as to enable the radiation controller 36 to set image capturing conditions, which are identical to the image capturing conditions set by the system controller 24.

Then, in step S1 shown in FIG. 4, during a time interval from time t1 to time t2 shown in FIG. 5, the leak quantity detector 114 of the cassette controller 60 controls the gate drive circuit 80 to turn off all of the TFTs 70 (see FIG. 3). In step S2, the leak quantity detector 114 sets the gain A1 of the amplifiers 84 to a level that is higher than the gain A2 thereof at the time that the electric signals are read out in step S6, as described later (A1>A2).

If the user then presses the radiation switch 38 (see FIG. 1), then in step S3 shown in FIG. 4, the radiation source 34 applies radiation 16 to the subject 14 at a prescribed dosage according to the image capturing conditions, for a prescribed exposure time during a time interval from time t2 to time t3, as shown in FIG. 5. Radiation 16 passes through the subject 14 to the radiation conversion panel 52 in the housing 44 (see FIGS. 2 and 3). The scintillator 54 emits visible light at an intensity depending on the intensity of the radiation 16. The photoelectric transducer elements 68 of the photoelectric transducer layer 56 convert the visible light into electric signals and store the electric signals as electric charges. A portion of the stored electric charges leaks into the signal lines 72 via the gates of the TFTs 70, which are in an off state.

While radiation 16 is being applied during the time interval from time t2 to time t3 shown in FIG. 5, the amount of electric charge stored respectively in the photoelectric transducer elements 68 increases over time. During a time interval from the time at which application of radiation 16 is completed until a time at which the electric signals start to be read out, i.e., a time interval from time t3 to time t6 shown in FIG. 5, the amount of electric charge, which depends on the applied dose of radiation 16, is stored respectively in the photoelectric transducer elements 68.

As shown in FIG. 5, the leak signals (leak currents), which leak from the photoelectric transducer elements 68 via the TFTs 70 into the signal lines 72, increase over time from the time at which radiation 16 starts to be applied, and the leak signals are kept at a constant level during the time interval from the time at which application of radiation 16 is completed until the time at which the electric signals start to be read out. Since, as described above, plural photoelectric transducer elements 68 are connected to each signal line 72 (see FIG. 3) through respective TFTs 70, each signal line 72 transmits the sum of the leak signals from the plural photoelectric transducer elements 68 to the signal readout circuit 82.

During a time interval from time t3 to time t4 in FIG. 5, the leak quantity detector 114 of the cassette controller 60 controls the signal readout circuit 82, so as to enable the amplifiers 84, which are set to the large gain A1, to amplify the leak signals, which are extremely low in level. The amplified leak signals are sampled and held by the sample and hold circuits 86. The sampled and held leak signals are successively supplied via the multiplexer 88 to the A/D converter 90. The A/D converter 90 converts the successively supplied leak signals into digital leak signals at a certain number of sampling, which is lower than the number of sampling according to the oversampling A/D conversion process, and outputs the digital leak signals to the leak quantity detector 114.

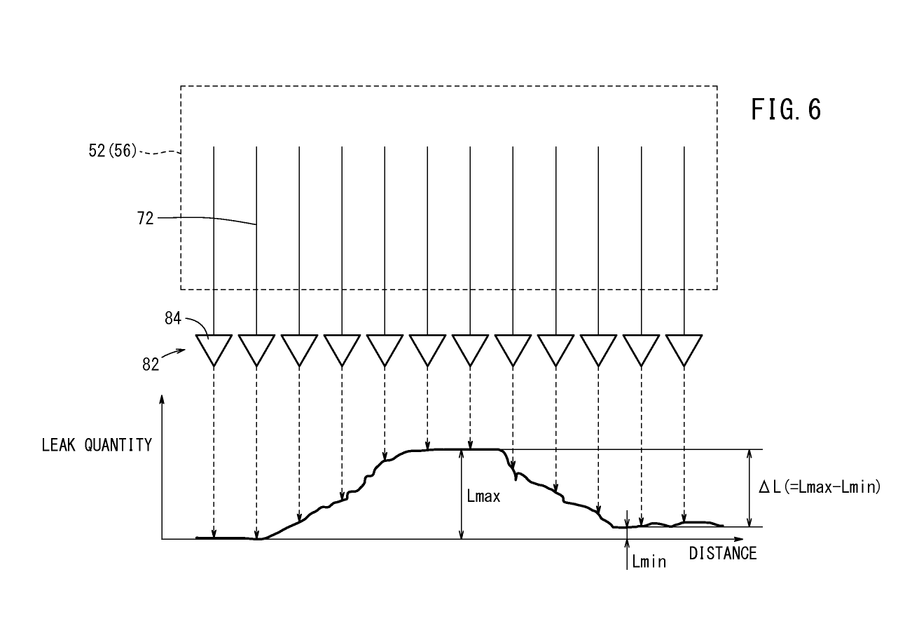

In step S4, the leak quantity detector 114 generates a profile (see FIG. 6) indicative of changes in the signal levels of the leak signals along the array of the signal lines 72, using the digital data of the leak signals from the respective signal lines 72, and stores the generated profile in the storage unit 110. Thereafter, the leak quantity detector 114 resets the amplifiers 84 from the gain A1 to the gain A2 at the time that the electric signals are read out.

FIG. 6 shows that signal levels of the leak currents (leak quantities), which leak from signal lines 72 disposed in a central area of the radiation conversion panel 52, are higher in level, whereas signal levels of the leak currents (leak quantities), which leak from signal lines 72 disposed in opposite end areas of the radiation conversion panel 52, are lower in level.

In step S5, at time t4 shown in FIG. 5, the oversampling controller 108 determines whether or not the oversampling A/D conversion process should be performed by the A/D converter 90, at the time that the electric charges (electric signals) are read out from the photoelectric transducer elements 68, so as to acquire a radiographic image depending on the electric signals, based on the signal levels of the leak signals, which are detected by the leak quantity detector 114.

The oversampling controller 108 may perform any one of the following determination processes.

(1) The storage unit 110 (see FIG. 3) stores a table representing a relationship between signal levels of leak signals, the amounts of electric charge stored in the photoelectric transducer elements 68, and the applied dosage of radiation 16.

The oversampling controller 108 refers to the above table in order to estimate the amounts of electric charge and the applied dose of radiation 16 depending on a maximum value Lmax (see FIG. 6) of the signal levels, according to the profile stored in the storage unit 110. Then, if the estimated amounts of electric charge and the applied dose of radiation 16 depending on the maximum value Lmax are relatively small, then since the signal levels of the electric signals are small, the oversampling controller 108 decides that a radiographic image with noise added thereto is acquired by performing an A/D conversion process at the number of sampling, which is lower than the number of sampling according to the oversampling A/D conversion process. Based on this decision, the oversampling controller 108 decides that the oversampling A/D conversion process should be performed while also determining the number of sampling thereof.

If the estimated amounts of electric charge and the applied dose of radiation 16, which depends on the maximum value Lmax, are relatively large, then since the signal levels of the electric signals are large, the oversampling controller 108 decides that a radiographic image with little noise added thereto is acquired, without performing the oversampling A/D conversion process. Based on this decision, the oversampling controller 108 cancels the oversampling A/D conversion process (i.e., stops the oversampling A/D conversion process) and decides that an A/D conversion process, at the number of sampling that is lower than the number of sampling according to the oversampling A/D conversion process, should be performed.

(2) The oversampling controller 108 calculates the difference ΔL (=Lmax−Lmin) between the maximum value Lmax and the minimum value Lmin of the signal levels according to the profile, and refers to the above table in order to estimate the difference between the amounts of electric charge and the difference between the applied doses, which depend on the calculated difference ΔL. Then, if the difference between the amounts of electric charge and the difference between applied doses, which depend on the calculated difference ΔL, are relatively small, then the oversampling controller 108 decides that radiation 16 has been applied at a small dose, and further decides that the oversampling A/D conversion process should be performed, while also determining the number of sampling thereof.

If the difference between the amounts of electric charge and the difference between the applied doses, which depend on the calculated difference ΔL, are relatively large, then the oversampling controller 108 decides that radiation 16 has been applied at a large dose and cancels the oversampling A/D conversion process, and further decides that an A/D conversion process at the number of sampling, which is lower than the number of sampling according to the oversampling A/D conversion process, should be performed.

According to determining process (2), the oversampling controller 108 calculates the difference ΔL between the maximum value Lmax and the minimum value Lmin of the signal levels according to the profile, while excluding therefrom data from those signal lines 72 from which leak signals of a substantially zero level are produced, i.e., signal lines 72 which are connected to photoelectric transducer elements 68 that are judged as not being irradiated with radiation 16. Therefore, if the difference ΔL is small, then the maximum value Lmax is of a signal level close to the minimum value Lmin, and hence the oversampling controller 108 decides that the radiation conversion panel 52 is irradiated with a small dose of radiation 16 overall.

If the difference ΔL is relatively small, then the radiographic image possibly is of a low gradation, and noise may increase or become visible if the gradation is expanded by performing image processing on the radiographic image, such as a gradation correcting process. Therefore, in this case, it is preferable to perform the oversampling A/D conversion process.

(3) The oversampling controller 108 performs a combination of the above determination processes (1) and (2).

More specifically, the oversampling controller 108 calculates the difference ΔL and refers to a table in order to estimate amounts of electric charge and an applied dose of radiation 16 depending on the maximum value Lmax. The oversampling controller 108 also calculates a difference between the amounts of electric charge and a difference between the applied doses depending on the calculated difference ΔL. Then, if the estimated amounts of electric charge and the applied dose of radiation 16 depending on the maximum value Lmax are small, and if the calculated difference between the amounts of electric charge and the difference between the applied doses of radiation 16 depending on the calculated difference ΔL are also relatively small, the oversampling controller 108 decides that the radiation 16 has been applied at a small dose. Based on this decision, the oversampling controller 108 decides that the oversampling A/D conversion process should be performed and determines the number of sampling thereof.

If the estimated amounts of electric charge and applied doses of radiation 16 depending on the maximum value Lmax are small, and if the calculated difference between the amounts of electric charge and the difference between applied doses of radiation 16 depending on the calculated difference ΔL are relatively large, then the oversampling controller 108 decides that the radiation 16 has been applied at a small dose. Based on this decision, the oversampling controller 108 decides that the oversampling A/D conversion process should be performed and determines the number of sampling thereof.

If the estimated amounts of electric charge and applied dose of radiation 16 depending on the maximum value Lmax are large, and if the calculated difference between the amounts of electric charge and the difference between applied doses depending on the calculated difference ΔL are also relatively large, then the oversampling controller 108 decides that the radiation 16 has been applied at a large dose. Based on this decision, the oversampling controller 108 cancels the oversampling A/D conversion process and decides that an A/D conversion process at the number of sampling, which is lower than the number of sampling according to the oversampling A/D conversion process, should be performed.

If the estimated amounts of electric charge and applied dose of radiation 16 depending on the maximum value Lmax are large, and if the calculated difference between the amounts of electric charge and the difference between applied doses depending on the calculated difference ΔL are relatively small, then the oversampling controller 108 decides that, even though radiation 16 has been applied at a large dose, gradation in the radiographic image is low because of the small difference ΔL, such that noise will tend to increase or become visible if the gradation is expanded by performing image processing, such as a gradation correcting process, on the radiographic image. Based on this decision, the oversampling controller 108 decides that the oversampling A/D conversion process should be performed and determines the number of sampling thereof.

Since the determination process (3) is a combination of determination processes (1) and (2), it is possible to determine the number of sampling more accurately based on the applied dose of radiation 16.

In step S6, after the number of sampling has been determined by the oversampling controller 108, the cassette controller 60 (i.e., the oversampling controller 108 thereof) controls the gate drive circuit 80 in order to apply a signal readout voltage to one of the gate lines 74. The gates of the TFTs 70, the gate electrodes of which are connected to the gate line 74, are opened, and the electric charges stored in the photoelectric transducer elements 68 connected to such TFTs 70, i.e., the electrons stored in the pin-type photoelectric transducer elements 68 shown in FIG. 3, are read as electric signals into the signal lines 72. The amplifiers 84 amplify the read electric signals, and the sample and hold circuits 86 sample and hold the amplified electric signals, and successively supply the amplified electric signals via the multiplexer 88 to the A/D converter 90.

In step S7, the A/D converter 90 converts the supplied electric signals into digital signals at the number of sampling determined by the oversampling controller 108. The digital electric signals represent a radiographic image, which is stored in the image memory 100 of the cassette controller 60 in step S8.

After completion of readout of the radiographic image represented by the electric signals from the photoelectric transducer elements 68 connected to one of the gate lines 74, the cassette controller 60 controls the gate drive circuit 80 to apply signal readout voltages successively to the other gate lines 74, in order to read out electric signals from the photoelectric transducer elements 68 that are connected to such gate lines 74. The process from steps S6 through S9 is repeated during the time interval from time t6 after time t5 until time 7 shown in FIG. 5, and until the readout of the radiographic image represented by the electric signals from all of the photoelectric transducer elements 68 connected the gate lines 74 is completed (step S9: YES).

In step S9, after the readout of the radiographic image, which is represented by electric signals from all of the photoelectric transducer elements 68, is completed, and after the radiographic image of the subject 14 has been stored in the image memory 100, the cassette controller 60 sends the radiographic image stored in the image memory 100 together with cassette ID information stored in the storage unit 110 from the communication unit 62 to the system controller 24 via a wireless communication link. The system controller 24 performs a predetermined image processing sequence on the received radiographic image, and sends the processed radiographic image to the display device 28 via a wireless communication link, whereupon the display device 28 displays the received radiographic image.

If a plurality of radiographic images of the subject 14 are to be captured, and if capturing of all of the radiographic images of the subject 14 is completed (step S11: YES), then the subject 14 is released from the image capturing base 12, and the operation sequence of the electronic cassette 20A is ended. If a plurality of radiographic images of the subject 14 are to be captured, and if capturing of all of the radiographic images of the subject 14 is not completed (step S11: NO), then control returns to step S1 in order to repeat the processes from steps S1 through S11.

### Advantages of the First Embodiment

With the electronic cassette 20A according to the first embodiment, as described above, the number of sampling of the A/D conversion process, which is carried out by the A/D converter 90, is determined based on the dose of radiation 16 applied to the radiation conversion panel 52. In the event that the A/D converter 90 performs an A/D conversion process at the determined number of sampling, therefore, the A/D conversion process is carried out depending on the applied dose of radiation 16. The electronic cassette 20A is thus able to acquire high-quality radiographic images while minimizing power consumption.

Since the signal levels of the leak signals become higher as the signal levels of electric signals, which depend on the applied dose of radiation 16, are higher, the applied dose of radiation 16 may be estimated from the signal levels of the leak signals, and the number of sampling may be determined based on the estimated applied dose of radiation 16, so that an A/D conversion process suitable for the applied dose of radiation 16 can be performed.

Consequently, if the signal levels of the leak signals are relatively low, then the oversampling controller 108 can control the A/D converter 90 to perform the oversampling A/D conversion process, whereas if the signal levels of the leak signals are relatively high, then the oversampling controller 108 can control the A/D converter 90 to cancel the oversampling A/D conversion process.

More specifically, if the signal levels of the leak signals are low, then the signal levels of the electric signals are low and the applied dose of radiation 16 is small. In this case, the oversampling A/D conversion process may be performed on the electric signals in order to reduce random noise in the digital electric signals.

Conversely, if the signal levels of the leak signals are high, then the signal levels of the electric signals are high and the level of random noise with respect to the electric signals is relatively low. In this case, inasmuch as radiographic images having a low noise level can be acquired without the need for the oversampling A/D conversion process, the oversampling A/D conversion process can be canceled, and hence an undue power consumption can be avoided. If the power consumption is adjusted in this manner depending on the applied dose of radiation 16, the total period of time required for the sampling process is shortened, and the service life of the power supply unit 94 is increased.

Since the electric signals are read out via the signal lines 72 into the signal readout circuit 82, the number of sampling may be determined according to a profile of leak currents along the array of signal lines 72, and the A/D conversion process may be performed on the electric signals according to the determined number of sampling, for thereby reducing random noise. As a result, high-quality radiographic images of the subject 14 can be acquired reliably.

Since the signal levels of the electric signals from the signal lines 72 may differ from each other, the number of sampling may be determined based on the difference ΔL, and the A/D conversion process may be performed on the read electric signals according to the determined number of sampling. As a result, high-quality radiographic images of the subject 14 can be acquired reliably.

Furthermore, the leak signals are lower in signal level than the electric signals. Therefore, upon acquiring the leak signals, the gain of the amplifiers 84 may be increased, and the leak signals may be amplified at an increased rate by the amplifiers 84, for thereby allowing the number of sampling to be determined with high accuracy.

If the scintillator 54 is made of CsI, then the electronic cassette 20A can capture radiographic images of the subject 14, even if radiation 16 is applied at a low dose. In this case, since the signal levels of the electric signals are reduced depending on the applied dose of radiation 16, the oversampling A/D conversion process is performed on the electric signals. Consequently, high-quality radiographic images of the subject 14 can be acquired using a scintillator 54 made of CsI.

According to the present embodiment, the oversampling controller 108 determines whether or not the oversampling A/D conversion process should be performed based on the applied dose of radiation 16 depending on signal levels of the leak signals. Therefore, even though the applied dose of radiation 16 differs in different image capturing modes, including a still image capturing mode, a moving image capturing mode, and an energy subtraction image capturing mode, the oversampling controller 108 can determine whether or not the oversampling A/D conversion process should be performed. More specifically, in the still image capturing mode and in the energy subtraction image capturing mode, since the applied dose of radiation 16 is large, the oversampling A/D conversion process may be canceled. In the moving image capturing mode, since the applied dose of radiation 16 is small, the oversampling A/D conversion process may be performed.

According to the first embodiment, therefore, a sampling process depending on the image capturing menu or the state of the electronic cassette 20A may be performed on the electric signals, which are generated by the photoelectric transducer elements 68.

### Second Embodiment

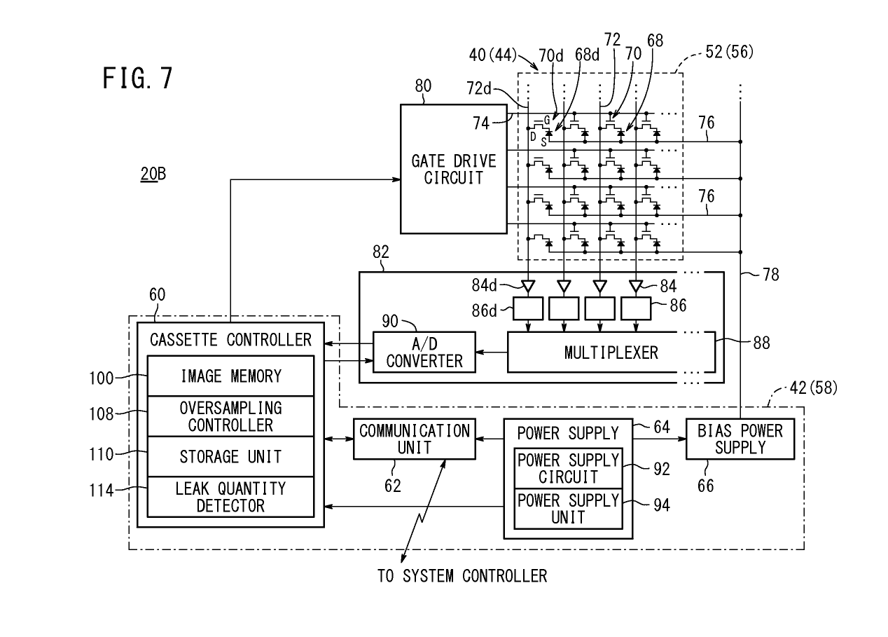

An electronic cassette 20B according to a second embodiment of the present invention will be described below with reference to FIG. 7. Components of the electronic cassette 20B according to the second embodiment, which are identical to those of the electronic cassette 20A according to the first embodiment, are denoted by identical reference characters and such features will not be described below. This also holds true for the third through fifth embodiments of the present invention, to be described later.

As shown in FIG. 7, the electronic cassette 20B according to the second embodiment differs from the electronic cassette 20A according to the first embodiment (see FIGS. 1 through 6), in that the electronic cassette 20B includes a column of TFTs 70d, the switching function of which does not operate normally, i.e., which is in an on state at all times. The column of TFTs 70d may be introduced at the time that the radiation conversion panel 52 is manufactured. Also, the amplifiers 84, 84d are not variable-gain amplifiers, but have a fixed gain equal to the gain A2. The amplifier 84d is connected to the column of TFTs 70d.

According to the second embodiment, the photoelectric transducer elements and the signal line, which are connected to the TFTs 70d that are in the on state at all times, are referred to as “photoelectric transducer elements 68d” and a “signal line 72d” respectively. The amplifier and the sample and hold circuit, which process the leak signals that leak from the photoelectric transducer elements 68d via the TFTs 70d and the signal line 72d, are referred to as an “amplifier 84d” and a “sample and hold circuit 86d” respectively.

The photoelectric transducer elements 68d and the signal line 72d, which are connected to the TFTs 70d, are components (defects) that do not contribute to detecting and reading of electric signals from the photoelectric transducer elements 68d. Normally, these components are not used to capture radiographic images of the subject 14.

According to the second embodiment, if such defects occur in the radiation conversion panel 52, the leak quantity detector 114 detects only leak signals that leak into the signal readout circuit 82 from the signal line 72d connected to the TFTs 70d.

Since the above defects are used as components that are dedicated to the detection of leak signals, all of the components of the electronic cassette 20B can be used effectively, and other components, which are dedicated to the detection of leak signals, do not need to be added.

Furthermore, inasmuch as the TFTs 70d are in the on state at all times during application of radiation 16 to the radiation conversion panel 52, electric charges, which are converted from visible light by the photoelectric transducer elements 68d, leak as leak signals via the TFTs 70d and the signal line 72d into the signal readout circuit 82 (refer to step S12 in FIG. 4). The signal levels of the leak signals, which are output via the signal line 72d to the amplifier 84d, are roughly the same as the signal levels of the electric signals that are output via a single signal line 72 to the corresponding amplifier 84. Therefore, in the present embodiment, the signal levels of the leak signals are much higher than the signal levels of the leak signals that leak via the single signal line 72 according to the first embodiment.

In the second embodiment, the amplifiers 84, 84d do not need to be variable-gain amplifiers, and hence the leak quantity detector 114 can accurately detect leak signals that leak via the signal line 72d. Therefore, according to the second embodiment, step S2 shown in FIG. 4 is not required.

The defects referred to above are not used to capture radiographic images of the subject 14. Consequently, the photoelectric transducer elements 68d appear as defective pixels in the captured radiographic image. The system controller 24 may perform a known image correcting process on such a captured radiographic image, using digital data from the photoelectric transducer elements 68, which serve as pixels around the defective pixels.

In the second embodiment, the above defects are introduced at the time that the radiation conversion panel 52 is manufactured. However, at the time that the radiation conversion panel 52 is manufactured, the above defects may actively be produced intentionally, as components which are dedicated to the detection of leak signals.

### Third Embodiment

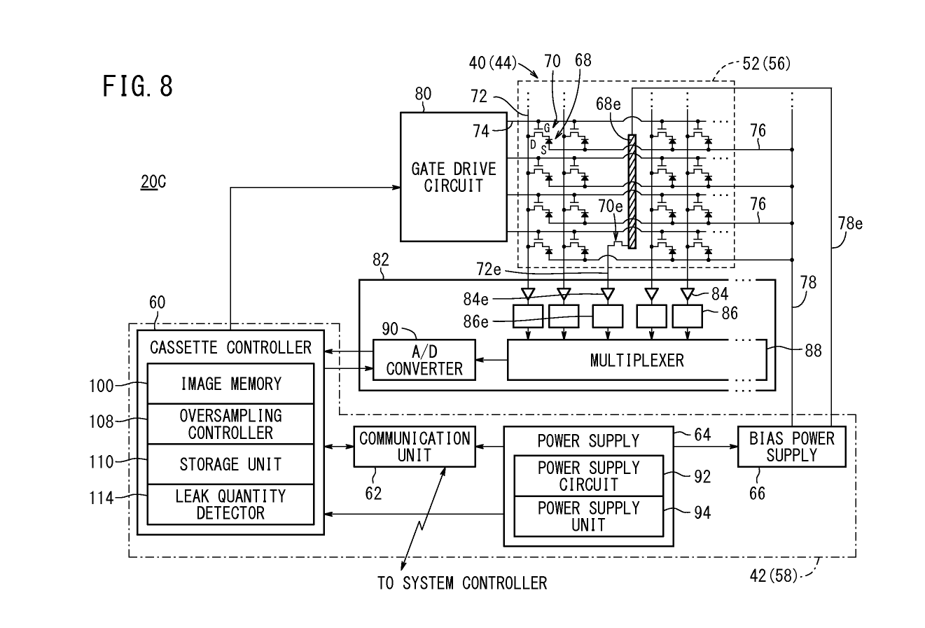

An electronic cassette 20C according to a third embodiment of the present invention will be described below with reference to FIG. 8.

As shown in FIG. 8, the electronic cassette 20C according to the third embodiment differs from the electronic cassettes 20A, 20B according to the first and second embodiments, in that the photoelectric transducer layer 56 includes a photoelectric transducer element 68e, which has a size corresponding to an area formed by the plurality of photoelectric transducer elements 68. Also, the photoelectric transducer element 68e has an anode, which is connected to the bias power supply 66 through a common line 78e, and a cathode, which is connected to the multiplexer 88 through a TFT 70e and is in an on state at all times, and a signal line (i.e., a leak signal output line 72e). The photoelectric transducer element 68e further comprises an amplifier 84e having a fixed gain, which is equal to the gain A2, and a sample and hold circuit 86e.

The leak quantity detector 114 may detect only a leak signal that leaks from the signal line 72e into the signal readout circuit 82 (refer to step S12 in FIG. 4). More specifically, according to the third embodiment, unlike the second embodiment, the common line 78e, the photoelectric transducer element 68e, the TFT 70e, the signal line 72e, the amplifier 84e, and the sample and hold circuit 86e, which are dedicated to the detection of leak signals, are positively included within the electronic cassette 20C. Although such components serve as components dedicated to the detection of leak signals, the components are capable of detecting leak signals regardless of whether electric signals are being read out. As described above, the size of the photoelectric transducer element 68e corresponds to the area formed by the plurality of photoelectric transducer elements 68. Consequently, upon application of radiation 16, the level of leak signals that leak from the photoelectric transducer element 68e is high enough for the leak quantity detector 114 to easily detect the leak signals.

Operations of the photoelectric transducer element 68e, the TFT 70e, the signal line 72e, the amplifier 84e, and the sample and hold circuit 86e according to the third embodiment are substantially the same as the operations of the photoelectric transducer element 68d, the TFT 70d, the signal line 72d, the amplifier 84d, and the sample and hold circuit 86d according to the third embodiment, and such operations will not be described in detail below.

### Fourth Embodiment

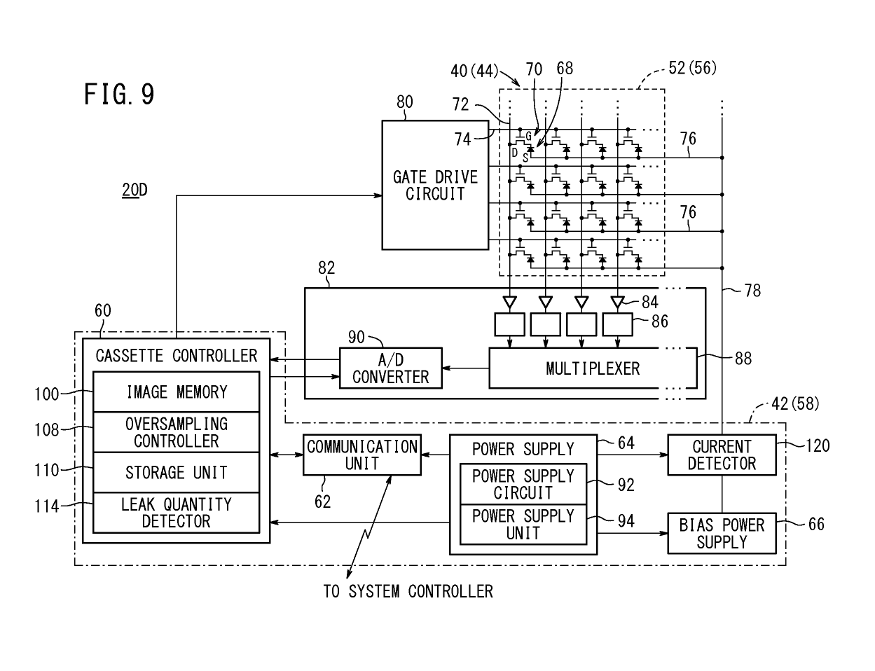

An electronic cassette 20D according to a fourth embodiment of the present invention will be described below with reference to FIG. 9.

As shown in FIG. 9, the electronic cassette 20D according to the fourth embodiment differs from the electronic cassettes 20A, 20B and 20C according to the first, second and third embodiments, in that the electronic cassette 20D includes a current detector 120 for detecting current (bias current) that flows through the common line 78.

The current detector 120 converts the bias current that flows through the common line 78 into a voltage value, so as to detect the voltage value by measuring the voltage across a resistor having a given resistance value. The resistor may be connected in series to the common line 78, for example.

According to the fourth embodiment, the leak quantity detector 114 regards a change in the voltage value, which is detected by the current detector 120, as representative of a change in the signal (leak signal) that leaks from the radiation conversion panel 52 into the common line 78, at the time that the radiation conversion panel 52 is irradiated with radiation 16. The oversampling controller 108 determines the number of sampling for the A/D conversion process, which is carried out by the A/D converter 90, based on the change in the voltage value.

More specifically, after step S1 shown in FIG. 4, as indicated by the broken lines, control proceeds to step S3 in which radiation 16 is applied. Then, in step S12, the current detector 120 detects a voltage value, which depends on a bias current that flows through the common line 78.

Since the reverse bias voltage (negative voltage) is applied to the anode electrodes of the photoelectric transducer elements 68, a potential gradient is developed in each of the photoelectric transducer elements 68. Thus, in the event that visible light is applied to the photoelectric transducer elements 68, electron and hole pairs are generated in the photoelectric transducer elements 68. Among the electron and hole pairs, the electrons move toward the cathode electrodes at a higher potential according to a potential gradient. Since the gates of the TFTs 70 are closed, the electrons are stored in the vicinity of the cathode electrodes. Therefore, an amount of electrons, which depends on the amount of visible light, is stored in each of the photoelectric transducer elements 68.

Among the electron and hole pairs, the holes move toward the anode electrodes at a lower potential according to the potential gradient, and the holes flow through the anode electrodes into the bias lines 76. The holes, which flow from the photoelectric transducer elements 68 and through the bias lines 76 and the common line 78, are detected as a bias current by the current detector 120.

More specifically, the amount of holes, which is identical to the amount of electrons that are stored in the photoelectric transducer elements 68 depending on the amount of applied visible light, flows through the bias lines 76. Currents that flow through the bias lines 76 are collected in the common line 78, and flow through the common line 78 toward the current detector 120.

Before radiation 16 is applied, ideally, no current flows through the bias lines 76 and the common line 78. However, actually, since dark currents are generated in the photoelectric transducer elements 68, the current detector 120 detects a small amount of current.

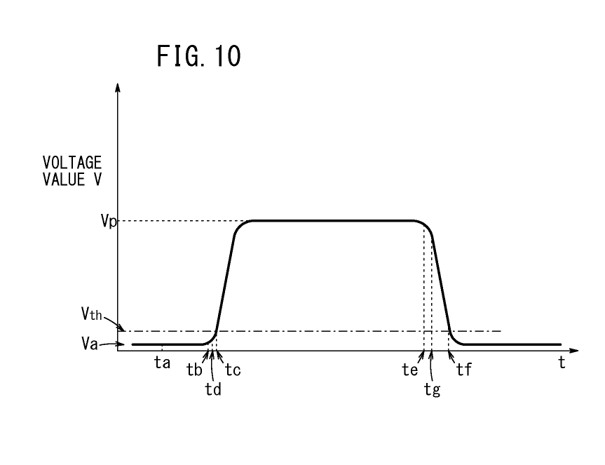

According to the fourth embodiment, as described above, the current detector 120 converts current that flows through the common line 78 into a voltage value, and then outputs the voltage value. Therefore, even before radiation 16 is applied, a small voltage Va, which is not nil, is applied from the current detector 120 to the leak quantity detector 114 of the cassette controller 60, at time to shown in FIG. 10, i.e., somewhere between time t1 and time t2 shown in FIG. 5.

Upon initial application of radiation 16 from the radiation source 34, electron and hole pairs are generated in each of the photoelectric transducer elements 68, and holes are carried via the bias lines 76 and the common line 78 to the current detector 120. Therefore, the voltage value V output from the current detector 120 starts to rise at time tb shown in FIG. 10. Thus, upon the voltage value V output from the current detector 120 beginning to rise, the leak quantity detector 114 detects the start of application of radiation 16. The leak quantity detector 114 may detect the start of application of radiation 16 at time tc, at which the voltage value V exceeds a predetermined threshold value Vth. Alternatively, the leak quantity detector 114 may detect the start of application of radiation 16 at time td, at which a time differential of the voltage value V exceeds a predetermined threshold value.

If the radiation source 34 stops applying radiation 16, electron and hole pairs stop being generated in each of the photoelectric transducer elements 68, and no holes are supplied to the bias lines 76. Therefore, the voltage value V output from the current detector 120 starts to fall at time to shown in FIG. 10. According to the fourth embodiment, the leak quantity detector 114 may detect the end of application of radiation 16 by detecting that the voltage value V output from the current detector 120 begins to fall.

The leak quantity detector 114 may detect the end of application of radiation 16 at time tf, at which the voltage value V becomes lower than the predetermined threshold value Vth. Alternatively, the leak quantity detector 114 may detect the end of application of radiation 16 at time tg, at which a time differential of the voltage value V negatively exceeds a predetermined negative threshold value. It is assumed hereinbelow that radiation 16 starts being applied at time tc and stops being applied at time tf.

As described above, electron and hole pairs are generated in the photoelectric transducer elements 68 in proportion to the intensity of the applied visible light. Also, holes depending on the intensity of the applied visible light flow from the photoelectric transducer elements 68 into the bias lines 76. Therefore, the total dose of radiation 16 applied to the electronic cassette 20D from the start of application of radiation 16 until the end of application of radiation 16 can be calculated by measuring the total amount of current that has flowed through the common line 78.

According to the fourth embodiment, the oversampling controller 108 has a peak holding function, which serves to calculate the dose of radiation 16 with greater ease based on the detected result from the peak quantity detector 114. In accordance with such a peak holding function, the oversampling controller 108 calculates the applied dose of radiation 16 based on a time interval (tf−tc) between the start and end of application of radiation 16, and the peak value of the current, which flows through the common line 78 and is detected by the current detector 120.

More specifically, the oversampling controller 108 detects a peak value Vp of the voltage detected from time tc to time tf, and calculates an approximate value M for the dose of radiation 16 applied to the electronic cassette 20D, based on a value that is produced by multiplying the peak value Vp by a value produced by subtracting a constant α from the time interval (tf−tc) between the start and end of application of radiation 16, according to the following equation (1).

M=a×Vp×(tf−tc−α) (1)

where “a” also represents a constant.

The approximate value M of the applied dose of radiation 16 is determined as a value proportional to the area of a rectangular shape, which approximates the voltage value V from a rising edge after time tc in FIG. 10 to a falling edge before time tf in FIG. 10. The approximate value M can be calculated simply by detecting the peak value Vp.

Alternatively, an integrating circuit or the like may be used to calculate an integrated value of the voltage value V from time tc to time tf in FIG. 10 (or a value produced by subtracting a constant value corresponding to noise from the voltage value V). The dose of radiation 16 applied to the electronic cassette 20D may be calculated based on the integrated value of the voltage value V. Such an alternative process makes it possible to calculate the dose of radiation 16 more accurately.

In order to remove noise more appropriately, the integrating circuit may incorporate a bandpass filter, which only passes data within a prescribed frequency band, while attenuating and blocking data in other frequency bands. A voltage value corresponding to the current value output from the current detector 120 may pass through the bandpass filter and be integrated, whereby the dose of radiation 16 applied to the electronic cassette 20D is calculated based on an integrated value of the voltage.

Based on the calculated dose of radiation 16, which may include the approximate value M, the oversampling controller 108 determines the number of sampling for the A/D conversion process of the A/D converter 90, and controls the A/D converter 90 in order to convert electric signals into digital signals at the determined number of sampling (step S5 in FIG. 4).

With the electronic cassette 20D according to the fourth embodiment, as described above, the leak quantity detector 114 detects a change in current (a change in the voltage value V), which is generated upon the radiation conversion panel 52 being irradiated with radiation 16, as a signal that leaks from the radiation conversion panel 52 (leak current). In other words, attention is paid to the fact that the current is changed by application of radiation 16 to the radiation conversion panel 52. A change in (the voltage value V depending on) the current is regarded as a change in the signal level of the leak signal, and is detected, thereby efficiently detecting the voltage value V. Thus, the electronic cassette 20D according to the fourth embodiment offers the same advantages as the electronic cassette 20A according to the first embodiment.

### Fifth Embodiment

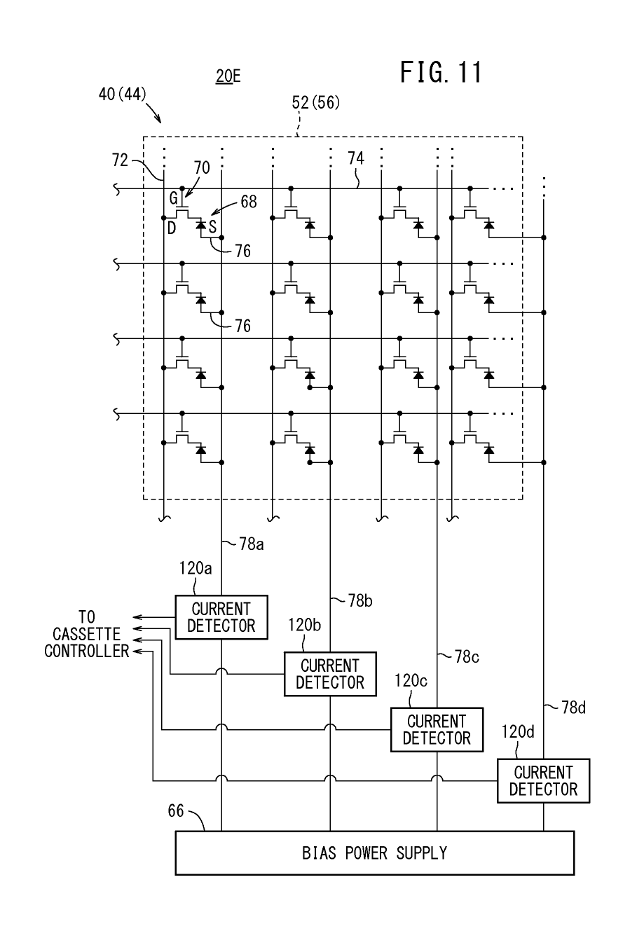

An electronic cassette 20E according to a fifth embodiment of the present invention will be described below with reference to FIG. 11.

As shown in FIG. 11, the electronic cassette 20E according to the fifth embodiment differs from the electronic cassette 20D according to the fourth embodiment, in that common lines (bias lies) 78a through 78d are connected to respective columns of photoelectric transducer elements 68, and current detectors 120a through 120d are connected respectively to the common lines 78a through 78d.

The current detectors 120a through 120d detect voltage values V (see FIG. 10) on the respective common lines 78a through 78d, and output the detected voltage values V to the cassette controller 60. With this arrangement, it is possible to accurately reduce random noise in the electric signals from the photoelectric transducer elements 68.

Although certain preferred embodiments of the present invention have been shown and described in detail, it should be understood that various changes and modifications may be made to the embodiments without departing from the scope of the invention as set forth in the appended claims.

What is claimed is:

1. A radiographic image capturing apparatus comprising:
a radiation conversion panel for converting radiation into a radiographic image;
an A/D converter for performing an A/D conversion process on an image signal depending on the radiographic image output from the radiation conversion panel; and
a number-of-sampling determiner for determining a number of times of sampling for the A/D conversion process performed by the A/D converter, based on the dose of the radiation that is applied to the radiation conversion panel.

2. The radiographic image capturing apparatus according to claim 1, further comprising:
a signal reader for reading out the image signal from the radiation conversion panel; and
a leak signal detector for detecting leak signals, which leak from the radiation conversion panel, from a time at which the radiation starts to be applied to the radiation conversion panel until a time at which the image signal is read out from the radiation conversion panel by the signal reader,
wherein the number-of-sampling determiner estimates the dose of the radiation based on the signal levels of the leak signals, which are detected by the leak signal detector, and determines the number of times of sampling based on the estimated dose.

3. The radiographic image capturing apparatus according to claim 2, wherein if the signal levels of the leak signals are relatively low, the number-of-sampling determiner controls the A/D converter to perform an oversampling A/D conversion process, and if the signal levels of the leak signals are relatively high, the number-of-sampling determiner controls the A/D converter to cancel the oversampling A/D conversion process.

4. The radiographic image capturing apparatus according to claim 2, wherein the radiation conversion panel comprises a scintillator for converting the radiation into visible light, and a photoelectric transducer layer for converting the visible light into the image signal;
the photoelectric transducer layer includes a matrix of photoelectric transducer elements for converting the visible light into the image signal, and switching elements for outputting the image signal from the photoelectric transducer elements to the signal reader, a plurality of scanning lines that are supplied with control signals for turning on the switching elements, and a plurality of signal lines extending across the scanning lines, which are supplied with the image signal output from the photoelectric transducer elements; and
the switching elements are connected to the scanning lines and to the signal lines.

5. The radiographic image capturing apparatus according to claim 4, wherein if the switching elements are in an off state, the leak signals are signals that leak from the photoelectric transducer elements via the switching elements and the signal lines into the signal reader.

6. The radiographic image capturing apparatus according to claim 5, wherein the leak signal detector detects the leak signals, which leak from the signal lines into the signal reader, and generates a profile representative of changes in the signal levels of the leak signals along the array of the signal lines; and
the number-of-sampling determiner determines the number of times of sampling based on the generated profile.

7. The radiographic image capturing apparatus according to claim 6, wherein the number-of-sampling determiner calculates a level of difference between maximum and minimum values of the signal levels in the profile, and determines the number of times of sampling based on the calculated level difference.

8. The radiographic image capturing apparatus according to claim 5, wherein the signal reader includes amplifiers connected between the signal lines and the A/D converter; and
if the leak signals leak from the signal lines into the signal reader, the amplifiers comprise variable-gain amplifiers for amplifying the leak signals with a gain that is higher than a gain for amplifying the image signal.

9. The radiographic image capturing apparatus according to claim 4, wherein if the switching elements include switching elements that are in an on state at all times, the leak signal detector detects only leak signals, which leak from signal lines connected to the switching elements that are in the on state at all times, into the signal reader.

10. The radiographic image capturing apparatus according to claim 4, wherein the photoelectric transducer layer includes other photoelectric transducer elements for converting the visible light into electric signals, and a leak signal output line for outputting the electric signals as the leak signals from the other photoelectric transducer elements to the signal reader; and
the leak signal detector detects only the leak signals that are output from the leak signal output line to the signal reader.

11. The radiographic image capturing apparatus according to claim 4, further comprising:
bias lines for supplying a bias voltage to the photoelectric transducer elements;
a bias power supply for applying the bias voltage via the bias lines to the photoelectric transducer elements; and
a current detector for detecting bias currents flowing through the bias lines,
wherein the leak signal detector detects a change in the bias currents, which occurs upon application of the radiation to the radiation conversion panel, as a change in the signal levels of the leak signals.

12. The radiographic image capturing apparatus according to claim 11, wherein the photoelectric transducer layer includes a plurality of bias lines extending across the scanning lines;
the current detector comprises a plurality of current detectors connected respectively to the bias lines; and
the leak signal detector detects a change in the bias currents, which flow through the bias lines, and which are detected by the current detectors, respectively, as a change in the signal levels of the leak signals.
